(19) United States
(12) Patent Application Publication
(10) Pub. No.: US 2021/0375487 A1
(43) Pub. Date: Dec. 2, 2021

(54) INFORMATION PROVIDING SYSTEM

(71) Applicant: INFORMATION SYSTEM ENGINEERING INC., Shinjuku-ku, Tokyo (JP)

(72) Inventor: Satoshi KURODA, Tokyo (JP)

(73) Assignee: INFORMATION SYSTEM ENGINEERING INC., Shinjuku-ku, Tokyo (JP)

(21) Appl. No.: 16/972,273

(22) PCT Filed: Oct. 31, 2019
(86) PCT No.: PCT/JP2020/029033

§ 371 Date: Dec. 4, 2020

(30) Foreign Application Priority Data
Oct. 31, 2019 (JP) 2019-199260

(51) Int. Cl. G16H 70/60; G16H 40/40

(52) U.S. Cl. CPC G16H 70/60; G16H 40/40

(57) ABSTRACT

An information providing system includes a first database that is built on machine learning, using a data structure including a plurality of items of training data including evaluation target information including image data of a medical device, and a second database that stores content IDs and reference information corresponding to the content IDs. The information providing system acquires data including first image data, in which a specific medical device and a specific identification label for identifying the specific medical device are photographed, selects a first meta ID based on the acquired data, selects a first content ID based on the first meta ID, and selects first reference information based on the first content ID. The information providing system outputs information including the first reference information, the first content ID, the first meta ID, and the evaluation target information.

### TECHNICAL FIELD

The present invention relates to an information providing system.

### BACKGROUND ART

In recent years, techniques for providing predetermined information to users from acquired images have been drawing attention. For example, in patent literature 1, an image of a crop is acquired from a wearable terminal, and a predicted harvest time is displayed on the wearable terminal's display panel as augmented reality.

The wearable terminal display system of patent literature 1 is a wearable terminal display system for displaying the harvest time of a crop on a display panel of a wearable terminal, and provided with an image acquiring means for acquiring an image of a crop that has entered the wearable terminal's field of view, an identifying means for analyzing the image and identifying the type of the crop, a selection means for selecting determination criteria based on the type, a determination means for analyzing the image based on the determination criteria and determining the color and size, a prediction means for predicting the harvest time of the crop based on the determination result, and a harvest time display means for displaying, on the wearable terminal's display panel, as augmented reality, the predicted harvest time of the crop that is visible through the display panel.

### CITATION LIST

### Patent Literature

Patent Literature 1: Japanese Patent No. 6267841

### SUMMARY OF INVENTION

### Problem to be Solved by the Invention

However, the wearable terminal display system disclosed in patent literature 1 specifies the type of a crop by analyzing images. Therefore, when a new relationship between an image and the crop is acquired, the wearable terminal display system has to learn this relationship anew, through machine learning. Consequently, when a new relationship is acquired, the time it takes for its updating poses the problem. Furthermore, on what basis certain information is output is not displayed, which raises the problem that the user cannot use, comfortably, information that is output.

The present invention has been made in view of the above-described problem, and it is therefore an object of the present invention to provide an information providing system that makes it possible to perform tasks in a short time, and use, comfortably, information that is output.

### Means for Solving the Problem

The information providing system according to the present invention is an information providing system for selecting reference information that is suitable when a user to perform a task related to a medical device works on the task, and includes acquiring means for acquiring acquired data including first image data, in which a specific medical device and a specific identification label for identifying the specific medical device are photographed, a first database that is built on machine learning, using a data structure including a plurality of items of training data including evaluation target information including image data, and meta IDs that link with the evaluation target information, meta ID selection means for looking up the first database and selecting a first meta ID, among the plurality of meta IDs, based on the acquired data, a second database that stores content IDs that link with the meta IDs, and the reference information corresponding to the content IDs, content ID selection means for looking up the second database and selecting a first content ID, among a plurality of content IDs, based on the first meta ID, reference information selection means for looking up the second database and selecting first reference information, among a plurality of items of reference information, based on the first content ID, and output means for outputting output information including the first reference information. The image data includes an image that shows the medical device and an identification label for identifying the medical device, and the output means outputs the output information including the first meta ID, the evaluation target information that has been used to select the first meta ID, and the first content ID that has been used to select the first reference information.

The information providing system according to the present invention is an information providing system for selecting reference information that is suitable when a user to perform a task related to a nursing care device works on the task, and includes acquiring means for acquiring acquired data including first image data, in which a specific nursing care device and a specific identification label for identifying the specific nursing care device are photographed, a first database that is built on machine learning, using a data structure including a plurality of items of training data including evaluation target information including image data, and a meta ID that links with the evaluation target information, meta ID selection means for looking up the first database and selecting a first meta ID, among the plurality of meta IDs, based on the acquired data, a second database that stores content IDs that link with the meta IDs, and the reference information corresponding to the content IDs, content ID selection means for looking up the second database and selecting a first content ID, among a plurality of content IDs, based on the first meta ID, reference information selection means for looking up the second database and selecting first reference information, among a plurality of items of reference information, based on the first content ID, and output means for outputting output information including the first reference information. The image data includes an image that shows the nursing care device and an identification label for identifying the nursing care device, and the output means outputs the output information including the first meta ID, the evaluation target information that has been used to select the first meta ID, and the first content ID that has been used to select the first reference information.

### Advantageous Effects of Invention

According to the present invention, it is possible to perform a task in a short time, and use, comfortably, information that is output.

### BRIEF DESCRIPTION OF DRAWINGS

FIG. 1 is a schematic diagram to show an example of the configuration of an information providing system according to the present embodiment;

FIG. 2 is a schematic diagram to show an example of the use of the information providing system according to the present embodiment;

FIG. 3 is a schematic diagram to show examples of a meta ID estimation processing database and a reference database according to the present embodiment;

FIG. 4 is a schematic diagram to show an example of a data structure for machine learning according to the present embodiment;

FIG. 5 is a schematic diagram to show an example of first approval information stored in the meta ID estimation processing database according to the present embodiment;

FIG. 6 is a schematic diagram to show an example of first approval information stored in the reference database according to the present embodiment;

FIG. 7 is a schematic diagram to show an example of the configuration of an information providing device according to the present embodiment;

FIG. 8 is a schematic diagram to show examples of functions of the information providing device according to the present embodiment;

FIG. 9 is a flowchart to show an example of the operation of the information providing system according to the present embodiment;

FIG. 10 is a schematic diagram to show an example of output information that is output from the information providing system according to the present embodiment;

FIG. 11 is a schematic diagram to show a first example of variation of functions of the information providing device according to the present embodiment;

FIG. 12 is a schematic diagram to show a first example of the meta ID estimation processing database updated by an updating unit according to the present embodiment; and

FIG. 13 is a schematic diagram to show a second example of the meta ID estimation processing database updated by the updating unit according to the present embodiment.

### DESCRIPTION OF EMBODIMENTS

Hereinafter, examples of the information providing system according to an embodiment of the present invention will be described with reference to the accompanying drawings.

(Configuration of Information Providing System 100)

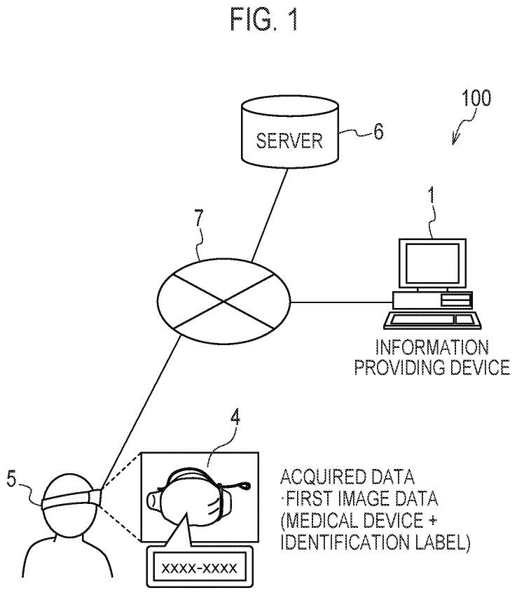

FIG. 1 is a block diagram to show an overall configuration of an information providing system 100 according to the present embodiment.

The information providing system 100 is used by users who use devices. Hereinafter, a case will be described in which these devices refers to medical devices 4. The information providing system 100 is used by users such as healthcare practitioners, including clinical engineers who use medical devices. The information providing system 100 is used primarily for medical devices 4, which are used by healthcare practitioners such as clinical engineers. The information providing system 100 selects, from acquired data carrying image data of a medical device 4, first reference information that is suitable when a user to perform a task related to the medical device works on the task. The information providing system 100 can provide, for example, a manual of the medical device 4 to the user, and, in addition, provide incident information related to the medical device 4 to the user, for example. By this means, the user can check the manual of the medical device 4, learn about the incidents related to the medical device 4, and so forth.

Furthermore, the information providing system 100 outputs, together with the first reference information, output information, which includes the first content ID that has been used to select the first reference information, the first meta ID, and the evaluation target information that has been used to select the first meta ID. Consequently, it is possible to display based on what kind of information the first reference information has been selected, so that it is possible to use the first reference information comfortably.

As shown in FIG. 1, the information providing system 100 includes an information providing device 1. The information providing device 1, for example, may be connected with at least one of a user terminal 5 and a server 6 via a public communication network 7.

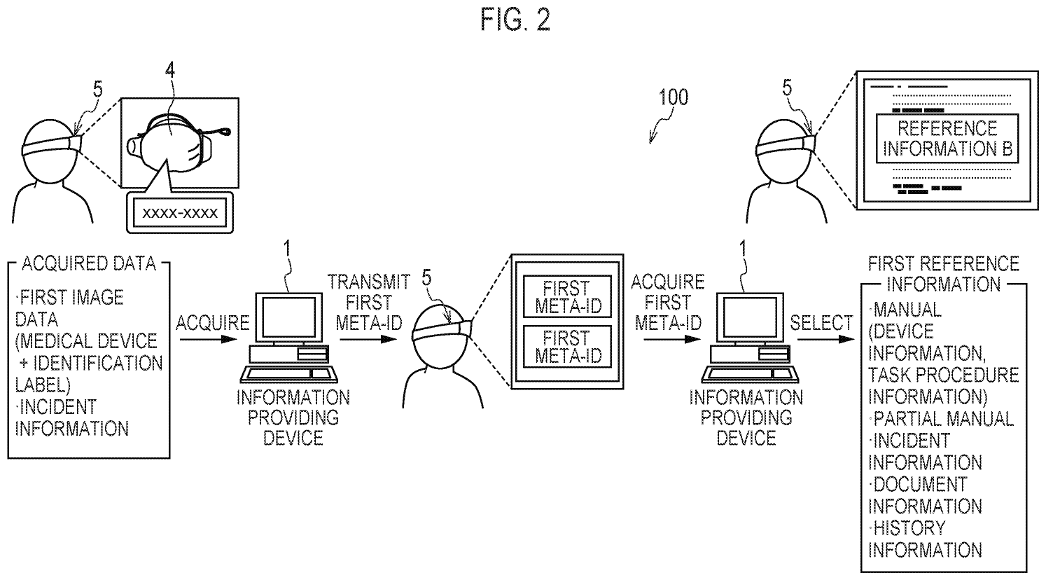

FIG. 2 is a schematic diagram to show an example of the use of the information providing system 100 according to the present embodiment. The information providing device 1 acquires data that carries first image data. The information providing device 1 selects the first meta ID based on the acquired data, and transmits the first meta ID to the user terminal 5. The information providing device 1 acquires the first meta ID from the user terminal 5. The information providing device 1 selects first reference information based on the first meta ID that is acquired, and transmits the first reference information to the user terminal 5. By this means, the user can have the first reference information, which carries the manual of the medical device 4 and/or the like.

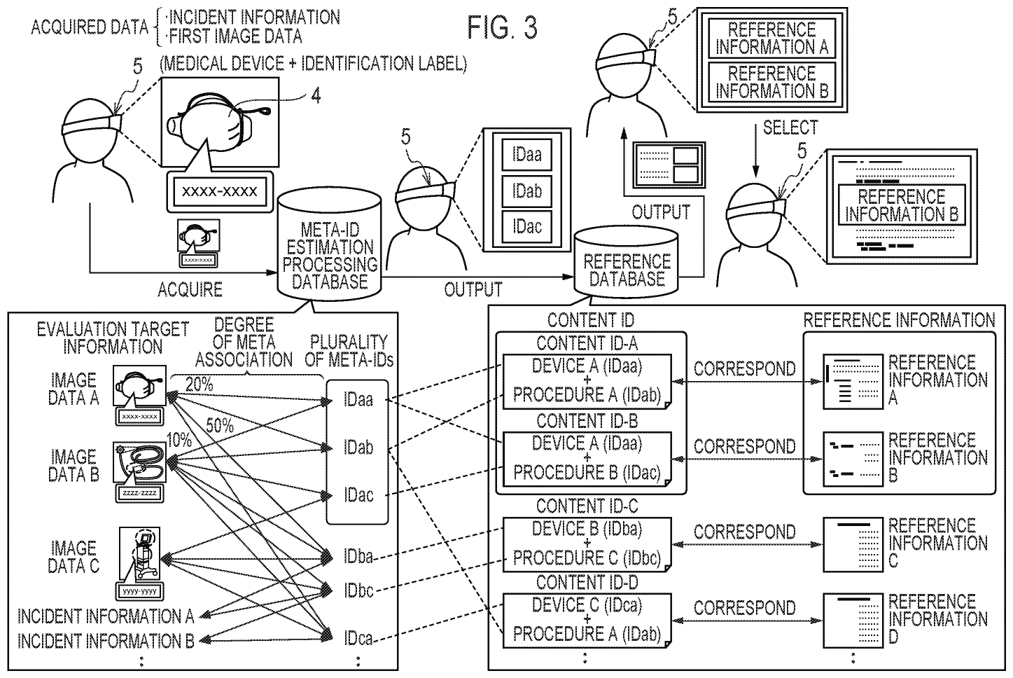

FIG. 3 is a schematic diagram to show examples of a meta ID estimation processing database and a reference database according to the present embodiment. The information providing device 1 looks up the meta ID estimation processing database (first database), and selects the first meta ID, among a plurality of meta IDs, based on the acquired data. The information providing device 1 looks up the reference database (second database), and selects the first content ID, among a plurality of content IDs, based on the first meta ID selected. The information providing device 1 looks up the reference database, and selects the first reference information, among a plurality of items of reference information, based on the first content ID selected.

The meta ID estimation processing database is built on machine learning, using a data structure for machine learning. The data structure for machine learning is used to build the meta ID estimation processing database, which a user to perform a task related to a medical device 4 uses to select reference information that is suitable when the user works on the task, and which is stored in a storage unit 104 provided in the information providing device 1 (computer).

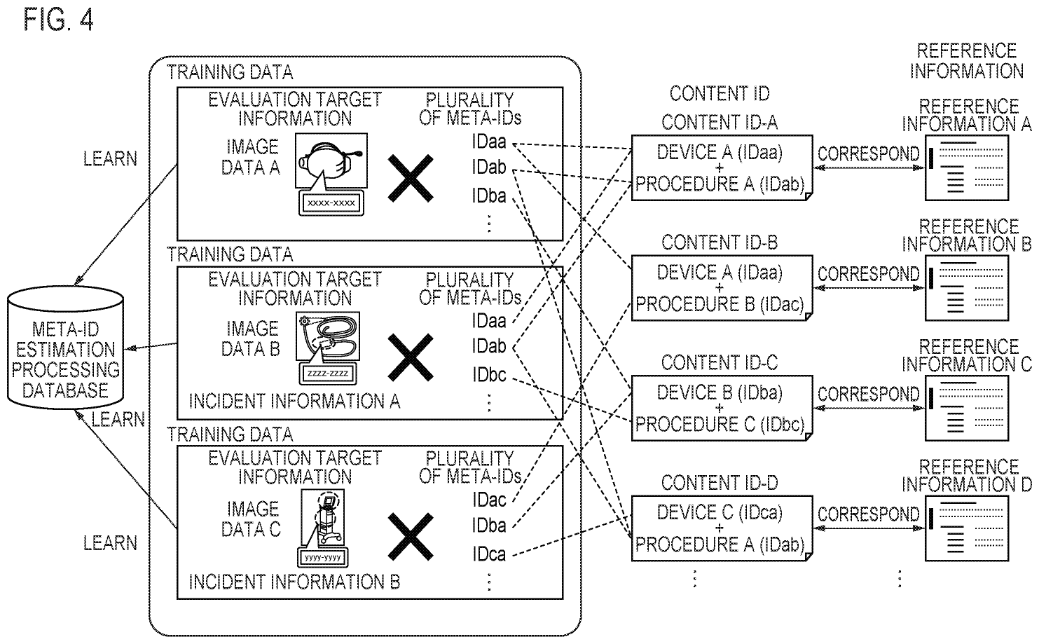

FIG. 4 is a schematic diagram to show an example of the data structure for machine learning according to the present embodiment. The data structure for machine learning includes carries a plurality of items of training data. These items of training data are used to build the meta ID estimation processing database on machine learning, which is implemented by a control unit 1 provided in the information providing device 1. The meta ID estimation processing database may be a pre-trained model that is built on machine learning using a data structure for machine learning.

Training data carries evaluation target information and meta IDs. The meta ID estimation processing database is stored in a storage unit 104.

The evaluation target information carries image data. The image data carries, for example, an image to show a medical device 4 and an identification label for identifying that medical device 4. The image may be a still image or a moving image. As for the identification label, one that consists of a character string of a product name, a model name, a reference number assigned so as to allow the user to identify the medical device 4, a one-dimensional code such as a bar code, a two-dimensional code such as a QR code (registered trademark) and so forth may be used. The evaluation target information may further carry incident information.

The incident information includes information about nearmiss accidents of the medical device 4, accident cases of the medical device 4 issued by administrative agencies such as the Ministry of Health, Labor and Welfare, and so forth. The incident information may include alarm information about the alarms that may be produced by the medical device 4. The incident information may be, for example, a file such as an audio file or the like, and may be a file such as an audio file of a foreign language translation corresponding to Japanese. For example, if one country's language is registered in audio format, a translated audio file in a foreign language corresponding to that registered audio file may be stored together.

The meta IDs consist of character strings and are linked with content IDs. The meta IDs are smaller than the reference information in volume. The meta IDs include, for example, an apparatus meta ID that classifies the medical device 4 shown in the image data, and a task procedure meta ID that relates to the task procedures of the medical device 4 shown in the image data. The meta IDs may also include an incident meta ID that relates to the incident information shown in the acquired data.

The acquired data carries first image data. The first image data is an image that is taken by photographing a specific medical device and a specific identification label for identifying that specific medical device. The first image data is, for example, image data that is photographed by the camera of the user terminal 5 or the like. The acquired data may further include incident information.

As shown in FIG. 3, the degrees of meta association between evaluation target information and meta IDs are stored in the meta ID estimation processing database. The degree of meta association shows how strongly evaluation target information links with a meta ID, and is expressed, for example, in percentage, or in three or more levels, such as ten levels, five levels, and so on. For example, referring to FIG. 3, “image data A” included in evaluation target information shows its degree of meta association with the meta ID “IDaa”, which is “20%”, and shows its degree of meta association with the meta ID “IDab”, which is “50%”. This means that “IDab” is more strongly linked with “image data A” than “IDaa” is.

The meta ID estimation processing database may have, for example, an algorithm that can calculate the degree of meta association. For example, a function (classifier) that is optimized based on evaluation target information, meta IDs, and the degrees of meta association may be used for the meta ID estimation processing database.

The meta ID estimation processing database is built by using machine learning, for example. As for the method of machine learning, for example, deep learning is used. The meta ID estimation processing database is, for example, built with a neural network, and, in that case, the degree of meta association may be represented by hidden layers and weight variables.

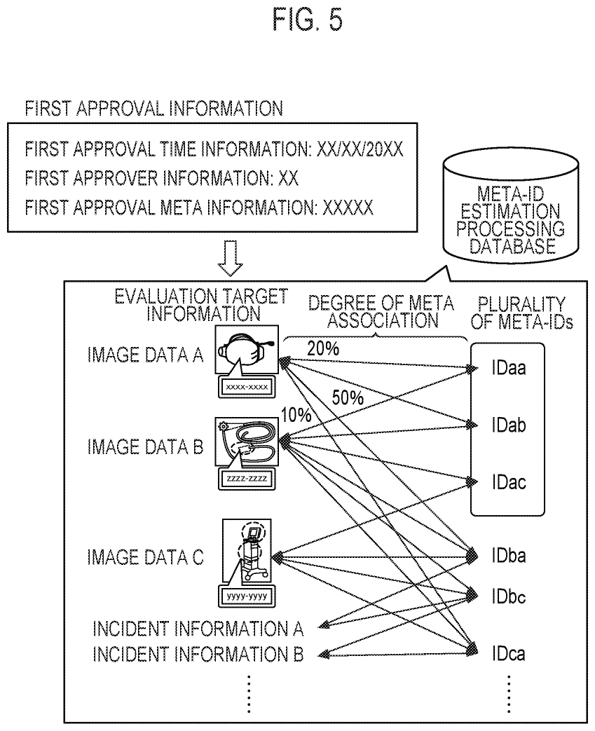

FIG. 5 is a schematic diagram to show an example of first approval information stored in the meta ID estimation processing database according to the present embodiment. The meta ID estimation processing database stores first approval information, which shows that evaluation target information and a meta ID have been approved. The first approval information includes at least one of first approval time information, which shows the time the evaluation target information and the meta ID were approved, first approver information, which shows the person who approved the evaluation target information and the meta ID, and first approval meta information, which shows the reason the evaluation target information and the meta ID were approved. The first approval time information and the first approver information may be formed with character string data. In the first approval meta information, the reason for the approval may be formed with character string data such as a comment. In the meta ID estimation processing database, first approval information, which shows that evaluation target information and a meta ID have been approved, may be stored.

As shown in FIG. 4, the reference database stores a plurality of content IDs and reference information. The reference database is stored in the storage unit 104.

A content ID consists of character strings, and is linked with one or more meta IDs. The content ID is smaller than the reference information in volume. The content ID includes, for example, a device ID that classifies the medical device 4 shown in the reference information, and a task procedure ID that relates to the task procedures of the medical device 4 shown in the reference information. The content ID may further include, for example, an incident ID that relates to the incident information of the medical device 4 shown in the reference information. The device ID is linked with a device meta ID in the meta IDs, and the task procedure ID is linked with a task procedure meta ID in the meta IDs. The incident ID is linked with an incident meta ID.

The reference information corresponds to content IDs. One item of reference information is assigned one content ID. The reference information includes, for example, information about a medical device 4. The reference information includes, for example, the manual, partial manuals, incident information, document information, history information and so forth, of the medical device 4. The reference information may have a chunk structure, in which meaningful information constitutes a chunk of a data block. The reference information may be a movie file. The reference information may also be an audio file, or may be an audio file of a foreign language translation corresponding to Japanese. For example, if one country's language is registered in audio format, a translated audio file in a foreign language corresponding to that registered audio file may be stored together.

The manual includes device information and task procedure information. The device information is information that classifies the medical device 4, and includes the specification, the operation and maintenance manual, and so forth. The task procedure information includes information about the task procedures of the medical device 4. The device information may be linked with the device ID, and the task procedure information may be linked with the task procedure ID. The reference information may include the device information and the task procedure information.

The partial manuals refer to predetermined portions of the manual that is divided. The partial manuals may divide the manual, for example, per page, per chapter, or per chunk structure, in which meaningful information constitutes a chunk of a data block. The manual and the partial manuals may be movies or audio data.

As mentioned earlier, the incident information includes information about nearmiss accidents of the medical device 4, accident cases of the medical device 4 issued by administrative agencies such as the Ministry of Health, Labor and Welfare, and so forth. Also, as mentioned earlier, the incident information may include alarm information about the alarms that may be produced by the medical device 4. In this case, the incident information may be linked, at least, either with the device ID or the task procedure ID.

The document information carries, for example, the specification, a research paper, a report and so on of the medical device 4.

The history information is information about, for example, the history of inspection, failures, and repairs of the medical device 4.

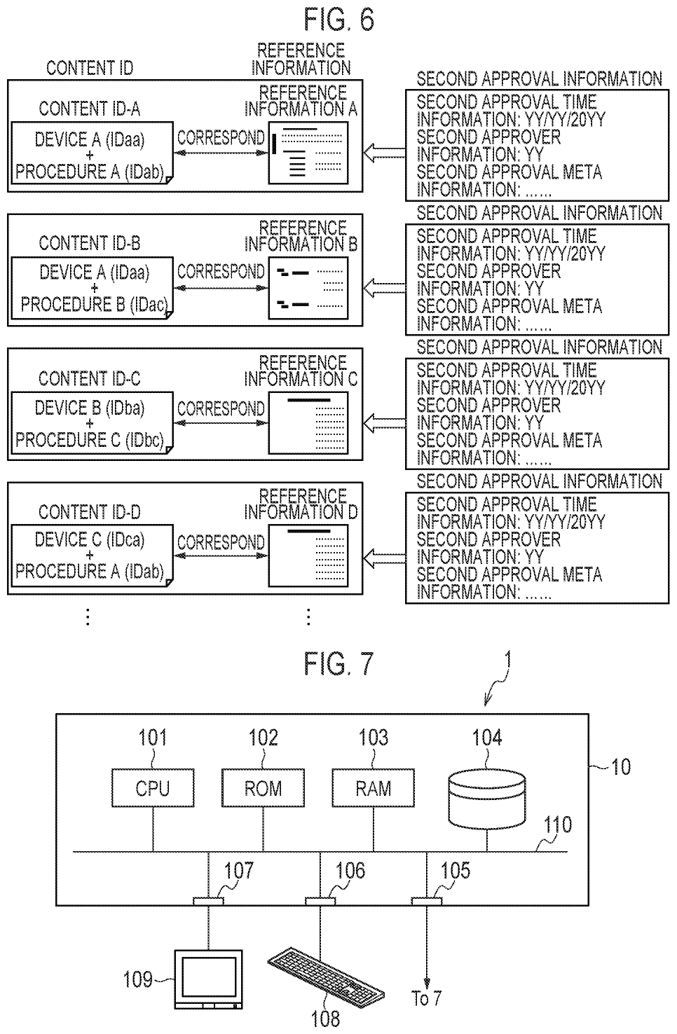

FIG. 6 is a schematic diagram to show an example of second approval information stored in the reference database according to the present embodiment. The reference database stores second approval information, which shows that a content ID and reference information are approved. The second approval information includes at least one of second approval time information, which shows the time a content ID and reference information were approved, second approver information, which shows the person who approved the content ID and the reference information, and second approval meta information, which shows the reason the content ID and the reference information were approved. The second approval time information and the second approver information may be formed with character string data. In the second approval meta information, the reason for the approval may be formed with character string data such as a comment.

<Information Providing Device 1>

FIG. 7 is a schematic diagram to show an example of the configuration of an information providing device 1. An electronic device such as a smartphone or a tablet terminal other than a personal computer (PC) may be used as the information providing device 1. The information providing device 1 includes a housing 10, a CPU 101, a ROM 102, a RAM 103, a storage unit 104 and I/Fs 105 to 107. The configurations 101 to 107 are connected by internal buses 110.

The CPU (Central Processing Unit) 101 controls the entire information providing device 1. The ROM (Read Only Memory) 102 stores operation codes for the CPU 101. The RAM (Random Access Memory) 103 is the task area for use when the CPU 101 operates. The storage unit 104 stores a variety of types of information such as a data structure for machine learning, acquired data, a meta ID estimation processing database, and a reference database. As for the storage unit 104, for example, an SSD (Solid State Drive) or the like is used, in addition to an HDD (Hard Disk Drive).

The I/F 105 is an interface for transmitting and receiving a variety of types of information to and from a user terminal 5 and/or the like, via a public communication network 7. The I/F 106 is an interface for transmitting and receiving a variety of types of information to and from an input part 108. For example, a keyboard is used as the input part 108, and the user to use the information providing system 100 inputs or selects a variety of types of information, control commands for the information providing device 1 and so forth, via the input part 108. The I/F 107 is an interface for transmitting and receiving a variety of types of information to and from an output part 109. The output part 109 outputs a variety of types of information stored in the storage unit 104, the state of processes in the information providing device 1, and so forth. A display may be used for the output part 109, and this may be a touch panel type, for example. In this case, the output part 109 may be configured to include the input part 108.

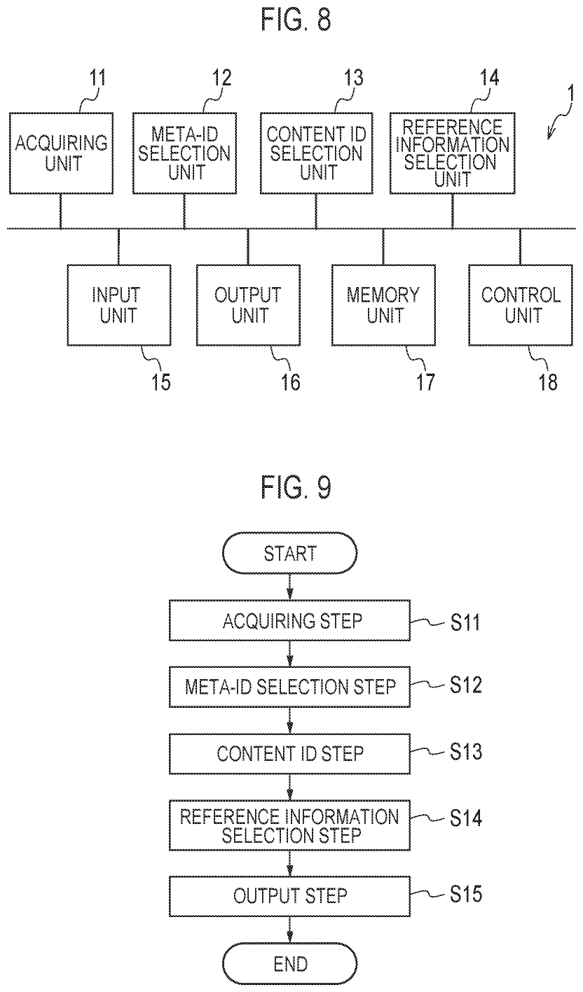

FIG. 8 is a schematic diagram to show examples of functions of the information providing device 1. The information providing device 1 includes an acquiring unit 11, a meta ID selection unit 12, a content ID selection unit 13, a reference information selection unit 14, an input unit 15, an output unit 16, a memory unit 17, and a control unit 18. Note that the functions shown in FIG. 8 are implemented when the CPU 101 runs programs stored in the storage unit 104 and elsewhere, by using the RAM 103 for the task area. Furthermore, each function may be controlled by, for example, artificial intelligence. Here, “artificial intelligence” may be based on any artificial intelligence technology that is known.

<Acquiring Unit 11>

The acquiring unit 11 acquires a variety of types of information such as acquired data. The acquiring unit 11 acquires the training data for building the meta ID estimation processing database.

<Meta ID Selection Unit 12>

The meta ID selection unit 12 looks up the meta ID estimation processing database, and selects first meta IDs, among a plurality of meta IDs, based on the acquired data. For example, when the meta ID estimation processing database shown in FIG. 3 is used, the meta ID selection unit 12 selects evaluation target information (for example, “image data A”) that is the same as or similar to the “first image data” included in the acquired image data. Also, when the meta ID estimation processing database shown in FIG. 3 is used, the meta ID selection unit 12 selects evaluation target information (for example, “image data B” and “incident information A”) that is the same as or similar to the “first image data” and “incident information” included in the acquired data.

As for the evaluation target information, information that partially or completely matches with the acquired data is selected, and, for example, similar information (including the same concept and/or the like) is used. The acquired data and the evaluation target information each include information of equal characteristics, so that the accuracy of the selection of evaluation target information can be improved.

The meta ID selection unit 12 selects one or more first meta IDs, from a plurality of meta IDs that link with the evaluation target information selected. For example, when the meta ID estimation processing database shown in FIG. 3 is used, the meta ID selection unit 12 selects, for example, the meta IDs “IDaa”, “IDab”, and “IDac”, as first meta IDs, among a plurality of meta IDs “IDaa”, “IDab”, “IDac”, “IDba”, and “IDca” linked with the “image data A” selected.

Note that the meta ID selection unit 12 may set a threshold for the degree of meta association, in advance, and select meta IDs that have higher degrees of meta association than that threshold, as first meta IDs. For example, if the degree of meta association of 50% or higher is the threshold, the meta ID selection unit 12 may select “IDab”, which shows a degree of meta association of 50% or higher, as a first meta ID.

<Content ID Selection Unit 13>

The content ID selection unit 13 looks up the reference database, and selects first content IDs, among a plurality of content IDs, based on the first meta IDs. For example, when the reference database shown in FIG. 3 is used, the content ID selection unit 13 selects content IDs (for example, “content ID-A”, “content ID-B”, etc.) linked with the first meta IDs “IDaa”, “IDab”, and “IDac” that are selected, as first content IDs. In the reference database shown in FIG. 3, “content ID-A” is linked with the meta IDs “IDaa” and “IDab”, and “content ID-B” is linked with the meta IDs “IDaa” and “IDac”. That is, the content ID selection unit 13 selects content IDs linked with any of the first meta IDs “IDaa”, “IDab”, and “IDac”, or combinations of these, as first content IDs. The content ID selection unit 13 uses a first meta ID as search query, and selects the results that match or partially match with the search query as first content IDs.

Also, if a device meta ID, among the first meta IDs selected, is to be linked with a device ID under a content ID, and a task procedure meta ID is to be linked with a task procedure ID under a content ID, the content ID selection unit 13 selects the content ID with the apparatus ID that links with the device meta ID or the content ID with the task procedure ID that links with the task procedure meta ID, as the first content ID.

<Reference Information Selection Unit 14>

The reference information selection unit 14 looks up the reference database, and selects first reference information, among a plurality of items of reference information, based on the first content ID. For example, when the reference database shown in FIG. 3 is used, the reference information selection unit 14 selects the reference information (for example, “reference information A”) that corresponds to the first content ID “content ID-A” selected, as first reference information.

<Input Unit 15>

The input unit 15 inputs a variety of types of information to the information providing device 1. The input unit 15 inputs a variety of types of information such as training data and acquired data via the I/F 105, and, additionally, inputs a variety of types of information from the input part 108 via, for example, the I/F 106.

<Output Unit 16>

The output unit 16 outputs output information, which includes a variety of types of information such as evaluation target information, first meta IDs, first content IDs, first reference information, first approval information, and second approval information, to the output part 109 and elsewhere. The output unit 16 transmits the first meta IDs and the output information to the user terminal 5 and elsewhere via, for example, the public communication network 7.

<Memory Unit 17>

The memory unit 17 stores a variety of types of information such as data structures for machine learning and acquired data, in the storage unit 104, and retrieves a variety of types of information stored in the storage unit 104 as needed. Furthermore, the memory unit 17 stores a variety of databases such as a meta ID estimation processing database, a reference database, a content database (described later), and a scene model database (described later), in the storage unit 104, and retrieves these databases stored in the storage unit 104 as needed.

<Control Unit 18>

The control unit 18 implements machine learning for building a first database by using data structures for machine learning. The control unit 18 implements machine learning using linear regression, logistic regression, support vector machines, decision trees, regression trees, random forest, gradient boosting trees, neural networks, Bayes, time series, clustering, ensemble learning, and so forth.

<Medical Device 4>

The medical devices 4, as used herein, include specially-controlled medical devices such as, for example, pacemakers, coronary stents, artificial blood vessels, PTCA catheters, central venous catheters, absorbable internal fixation bolts, particle beam therapy apparatus, artificial dialyzers, epidural catheters, infusion pumps, automatic peritoneal perfusion apparatus, artificial bones, artificial heart-lung machines, multi-person dialysate supply machines, apheresis apparatus, artificial respirators, programs and so forth (corresponding to the classifications of “class III” and “class IV” by GHTF (Global Harmonization Task Force)). The medical devices 4 also include controlled medical devices such as, for example, X-ray imaging apparatus, electrocardiographs, ultrasound diagnostic apparatus, injection needles, blood collection needles, vacuum blood collection tubes, infusion sets for infusion pumps, Foley catheters, suction catheters, hearing aids, home massagers, condoms, programs and so forth (corresponding to the classification of “class II” by GHTF). The medical devices 4 also include general medical devices such as, for example, enteral feeding sets, nebulizers, X-ray films, blood gas analyzers, surgical nonwoven fabrics, programs, and so forth (corresponding to the classification of “class I” by GHTF). The medical devices 4 not only include medical devices that are provided for in laws and regulations, but also include mechanical devices (beds, for example) and the like that are similar to medical devices in appearance and structures but are not provided for in laws and regulations. The medical devices 4 may be devices that are used at sites of medical practice such as hospitals, including medical information devices that store patients' medical records and electronic medical records, information devices that store information about the staff in hospitals, and so forth.

<User Terminal 5>

A user terminal 5 shows a terminal that a user to control a medical device 4 has. For example, the user terminal 5 may be HoloLens (registered trademark), which is one type of HMD (Head-Mounted Display). The user can check the task area, specific medical devices and so forth, through a display unit that shows the first meta IDs and the first reference information of the user terminal 5 in a transparent manner. This allows the user to check the situation in front of him/her, and also check the manual and so forth selected based on acquired data. Besides, electronic devices such as a mobile phone (mobile terminal), a smartphone, a tablet terminal, a wearable terminal, a personal computer, an IoT (Internet of Things) device, and, furthermore, any electronic device can be used to implement the user terminal 5. The user terminal 5 may be, for example, connected with the information providing device 1 via the public communication network 7, and, besides, may be connected directly with the information providing device 1, for example. The user may use the user terminal 5 to acquire the first reference information from the information providing device 1, and, besides, control the information providing device 1, for example.

<Server 6>

The server 6 stores a variety of types of information that have been described above. The server 6 stores, for example, a variety of types of information transmitted via the public communication network 7. The server 6 may store the same information as in the storage unit 104, for example, and transmit and receive a variety of types of information to and from the information providing device 1 via the public communication network 7. That is, the information providing device 1 may use the server 6 instead of the storage unit 104.

<Public Communication Network 7>

The public communication network 7 is, for example, an Internet network, to which the information providing device 1 and the like are connected via a communication circuit. The public communication network 7 may be constituted by a so-called optical fiber communication network. Furthermore, the public communication network 7 is not limited to a cable communication network, and may be implemented by a known communication network such as a wireless communication network.

(Example of Operation of Information Providing System 100)

Next, an example of the operation of the information providing system 100 according to the present embodiment will be described. FIG. 9 is a flowchart to show an example of the operation of the information providing system 100 according to the present embodiment.

<Acquiring Step S11>

First, the acquiring unit 11 acquires data (acquiring step S11). The acquiring unit 11 acquires the data via the input unit 15. The acquiring unit 11 acquires data that carries first image data, which is photographed by the user terminal 5, and incident information, which is stored in the server 6 or elsewhere. The acquiring unit 11 stores the acquired data in the storage unit 104 via, for example, the memory unit 17.

The acquired data may be generated by the user terminal 5. The user terminal 5 generates acquired data that carries first image data, in which a specific medical device and a specific identification label for identifying that specific medical device are photographed. The user terminal 5 may further generate incident information, or acquire incident information from the server 6 or elsewhere. The user terminal 5 may generate acquired data that carries the first image data and the incident information. The user terminal 5 transmits the generated acquired data to the information providing device 1. The input unit 15 receives the acquired data, and the acquiring unit 11 acquires that data.

<Meta ID Selection Step S12>

Next, the meta ID selection unit 12 looks up the meta ID estimation processing database, and selects the first meta ID, among a plurality of meta IDs, based on the acquired data (meta ID selection step S12). The meta ID selection unit 12 acquires the data acquired in the acquiring unit 11, and acquires the meta ID estimation processing database stored in the storage unit 104. The meta ID selection unit 12 may select one first meta ID for one item of acquired data, but may also select, for example, a plurality of first meta IDs for one item of acquired data. The meta ID selection unit 12 stores the selected first meta ID in the storage unit 104 via, for example, the memory unit 17.

The meta ID selection unit 12 transmits the first meta ID to the user terminal 5, and has the first meta ID displayed on the display unit of the user terminal 5. By this means, the user can check the selected first meta ID and the like. Note that the meta ID selection unit 12 may also have the first meta ID displayed on the output part 109 of the information providing device 1. The meta ID selection unit 12 may as well skip transmitting the first meta ID to the user terminal 5.

<Content ID Selection Step S13>

Next, the content ID selection unit 13 looks up the reference database, and selects the first content ID, among a plurality of content IDs, based on the first meta ID (content ID selection step S13). The content ID selection unit 13 acquires the first meta ID selected by the meta ID selection unit 12, and acquires the reference database stored in the storage unit 104. The content ID selection unit 13 may select one first content ID for one first meta ID, but may also select, for example, a plurality of first content IDs for one first meta ID. That is, the content ID selection unit 13 uses the first meta ID as a search query, and selects a result that matches or partially matches with the search query, as a first content ID. The content ID selection unit 13 stores the selected first content ID in the storage unit 104 via, for example, the memory unit 17.

<Reference Information Selection Step S14>

Next, the reference information selection unit 14 looks up the reference database, and selects first reference information, among a plurality of items of reference information, based on the first content ID (reference information selection step S14). The reference information selection unit 14 acquires the first content ID selected by the content ID selection unit 13, and acquires the reference database stored in the storage unit 104. The reference information selection unit 14 selects one item of first reference information corresponding to one first content ID. When the reference information selection unit 14 selects a plurality of first content IDs, the reference information selection unit 14 may select items of first reference information that correspond to these first content IDs respectively. By this means, a plurality of items of first reference information are selected. The reference information selection unit 14 stores the selected first reference information in the storage unit 104 via the memory unit 17, for example.

<Output Step S15>

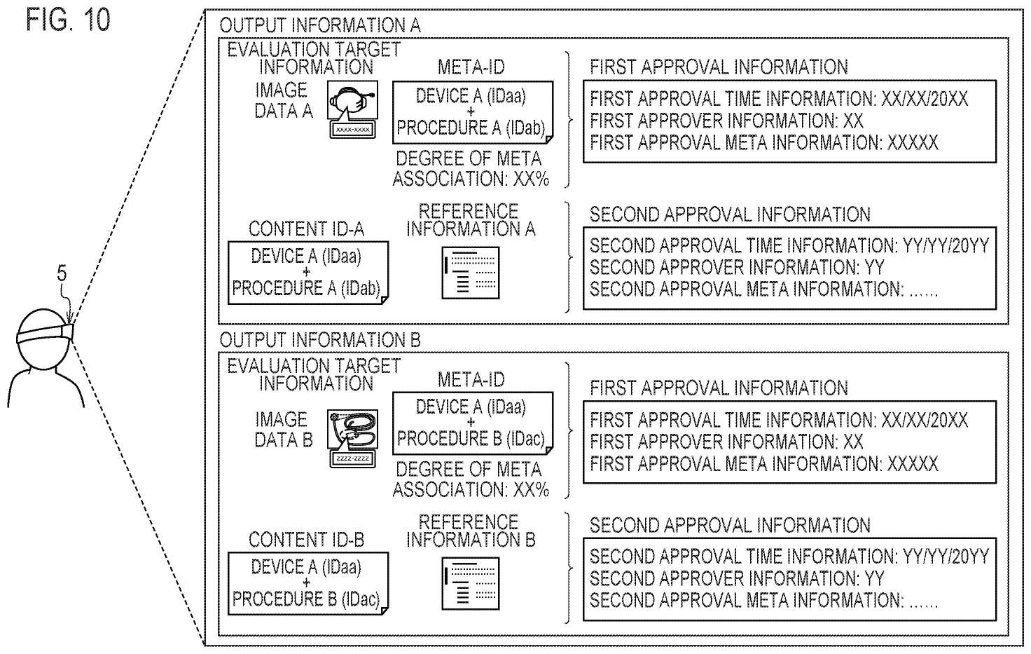

FIG. 10 is a schematic diagram to show an example of output information that is output from the information providing system according to the present embodiment. Next, the output unit 16 outputs output information, which includes the first reference information, to the output part 109 and the user terminal 5 (output step S15). Furthermore, the output unit 16 outputs output information, which includes the first content ID that was used to select first reference information, the first meta ID, and the evaluation target information that has been used to select the first meta ID.

The output unit 16 looks up the first database, and outputs output information, which includes first approval information related to the first meta ID and the evaluation target information that has been used to select the first meta ID. The output unit 16 looks up the second database and outputs output information, which includes second approval information related to the first reference information and the first content ID that has been used to select the first reference information.

The output unit 16 may also output information, which includes the first meta ID, the evaluation target information that has been used to select the first meta ID, and the degree of meta association between the first meta ID and the evaluation target information. Furthermore, the output unit 16 may also output output information, which includes the first reference information and the first content ID that has been used to select the first reference information.

For example, the output unit 16 transmits the first reference information to the user terminal 5 and elsewhere. The user terminal 5 displays one or a plurality of selected items of first reference information on the display unit. The user can select one or a plurality of items of first reference information from the one or the plurality of items of first reference information displayed. By this means, the user can learn one or a plurality of items of first reference information that carry the manuals and/or the like. In other words, one or more candidates for the first reference information suitable for the user are searched out from the image data of the medical device 4, and the user can make selection from the one or more searched candidates for the first reference information, so that this is very useful as a fieldwork solution for users who perform tasks related to medical devices 4 on site.

With this, the operation of the information providing system 100 according to the present embodiment is finished.

According to the present embodiment, meta IDs are linked with content IDs that correspond to reference information. It then follows that, when reference information is updated, it is only necessary to update the linking of the content ID corresponding to the reference information and meta IDs, or update the correspondence between the updated reference information and the content ID, so that it is not necessary to update the training data anew. By this means, it is not necessary to rebuild the meta ID estimation processing database when reference information is updated. Therefore, databases can be built in a short time when reference information is updated.

Furthermore, according to the present embodiment, when building the meta ID estimation processing database, machine learning can be executed using meta IDs that are smaller in volume than reference information. This makes it possible to build the meta ID estimation processing database in a shorter time than when machine learning is executed using reference information.

Also, according to the present embodiment, when searching for reference information, a meta ID, which is smaller in volume than image data, is used as a search query, and a content ID, which is smaller in volume than reference information, is returned as a result that matches or partially matches with the search query, so that the amount of data to communicate in the search process and the processing time can be reduced.

Furthermore, according to the present embodiment, when creating a system for searching for reference information by using machine learning based on data structures for machine learning, image data can be used as acquired data (input information) for use as a search keyword. Consequently, the user does not need to verbalize the information or a specific medical device that the user wants to search for, by way of inputting characters or voice, so that the search is possible without the knowledge of the information, the name of the medical device, and so on.

Furthermore, according to the present embodiment, together with first reference information, output information that includes the first content ID that has been used to select the first reference information, the first meta ID, and the evaluation target information that has been used to select the first meta ID are output. By this means, when the first reference information is output from the acquired data, the user can have the combination of evaluation target information and the first meta ID, and the combination of the first content ID and first reference information. That is, when first reference information is output from acquired data, it is possible to display based on what kind of information the first reference information has been selected. Consequently, the first reference information that is output can be used comfortably.

Furthermore, according to the present embodiment, first approval information related to the first meta ID and the evaluation target information that has been used to select the first meta ID, and second approval information related to the first reference information and the first content ID that has been used to select the first reference information, are output. By this means, when the first reference information is output from the acquired data, the user can learn that the combination of the evaluation target information and the first meta ID and the combination of the first content ID and the first reference information are approved. Consequently, the first reference information that is output can be used comfortably.

Furthermore, according to the present embodiment, the first approval information includes at least one of first approval time information, which shows the time the evaluation target information and the meta ID were approved, first approver information, which shows the person who approved the evaluation target information and the meta ID, and first approval meta information, which shows the reason the evaluation target information and the meta ID were approved, and the second approval information includes at least one of second approval time information, which shows the time the content ID and the reference information were approved, second approver information, which shows the person who approved the content ID and the reference information, and second approval meta information, which shows the reason the content ID and the reference information were approved.

This allows the user to learn, by himself/herself, when the combination of the evaluation target information and the first meta ID and the combination of the first content ID and the first reference information, which were used to select the first reference information, were approved. Consequently, for example, if the time of approval is too old, the user can learn that a variety of types of information need to be upgraded.

Also, the user can learn by whom the combination of the evaluation target information and the first meta ID and the combination of the first content ID and the first reference information, which were used to select the first reference information, were approved. Consequently, for example, the user can find the approver, and use, comfortably, the first reference information that is output.

Furthermore, the user can learn for what reason the combination of the evaluation target information and the first meta ID and the combination of the first content ID and the first reference information, which were used to select the first reference information, were approved. Consequently, for example, the user can learn the reason for approval, and use, comfortably, the first reference information that is output.

According to the present embodiment, device meta IDs are linked with device IDs, and task procedure meta IDs are linked with task procedure meta IDs. By this means, when selecting content IDs based on meta IDs, it is possible to narrow down the target range for the selection of content IDs. Consequently, the accuracy of selection of content IDs can be improved.

According to the present embodiment, a meta ID is linked with at least one content ID in a reference database, which, apart from the meta ID estimation processing database, stores a plurality of items of reference information and content IDs. Therefore, it is not necessary to update the reference database when updating the meta ID estimation processing database. Also, when updating the reference database, it is not necessary to update the meta ID estimation processing database. By this means, the task of updating the meta ID estimation processing database and the reference database can be performed in a short time.

According to the present embodiment, the reference information includes manuals for medical devices 4. By this means, the user can quickly learn the manual of the target medical device. Consequently, the time for searching for manuals can be reduced.

According to the present embodiment, the reference information includes partial manuals, which are predetermined portions of a manual of a medical device 4 that is divided. By this means, the user can learn the manual in a state in which parts of interest in the manual are narrowed down. Consequently, the time for searching for parts of interest in the manual can be shortened.

According to the present embodiment, the reference information further includes incident information of medical devices 4. By this means, the user can learn about the incident information. Therefore, the user can make quick reactions to nearmiss accidents or accidents.

According to the present embodiment, the evaluation target information further includes incident information of medical devices 4. This allows the incident information to be taken into account when selecting first meta IDs from the evaluation target information, so that the target range for the selection of first meta IDs can be narrowed down. Consequently, the accuracy of the selection of first meta IDs can be improved.

<First Example of Variation of Information Providing Device 1>

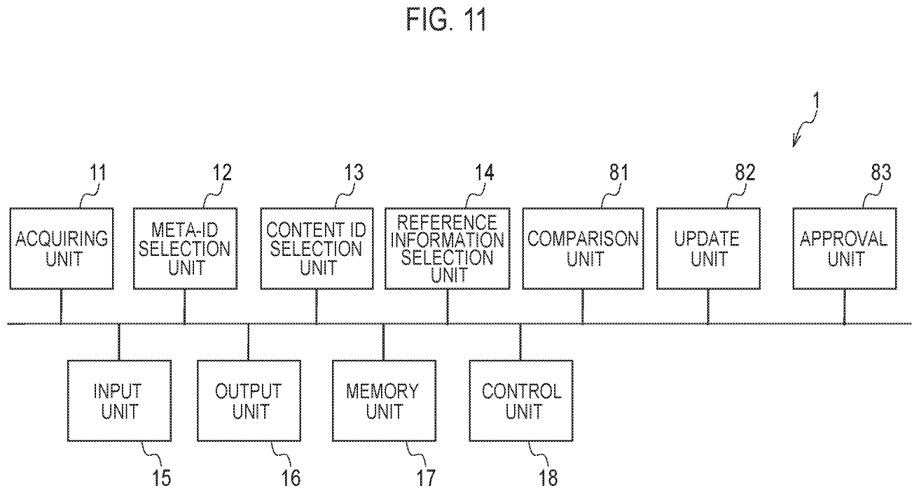

Next, the first example of a variation of the information providing device 1 will be described. This example of a variation is different from the above-described embodiment, primarily in that a comparison unit 81, an updating unit 82, and an approval unit 83 are additionally provided. Hereinafter, these differences will be primarily described. FIG. 11 is a schematic diagram to show the first example of variation of functions of the information providing device 1 according to the present embodiment. Note that the functions shown in FIG. 11 are implemented when the CPU 101 runs programs stored in the storage unit 104 and elsewhere, by using the RAM 103 for the task area. Furthermore, each function may be controlled by, for example, artificial intelligence. Here, “artificial intelligence” may be based on any artificial intelligence technology that is known.

<Comparison Unit 81>

The comparison unit 81 compares the acquired data with the evaluation target information. The comparison unit 81 determines whether the acquired data and the evaluation target information match or do not match.

<Updating unit 82>

When the acquired data and the evaluation target information compared in the comparison unit 81 do not match, the updating unit 82 updates the meta ID estimation processing database based on machine learning using the acquired data.

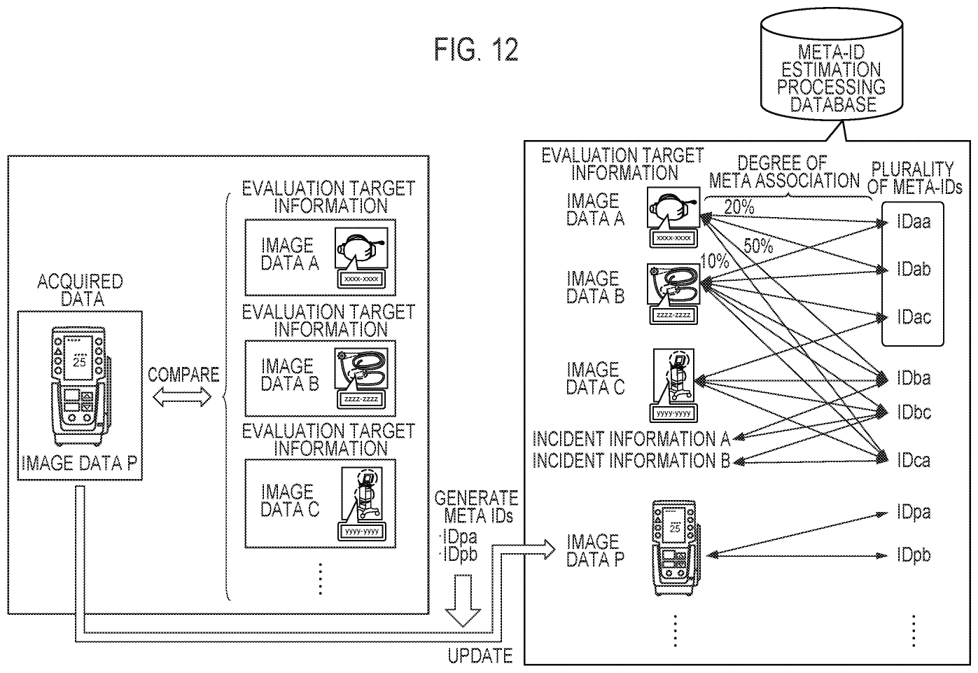

FIG. 12 is a schematic diagram to show the first example of the meta ID estimation processing database updated by the updating unit 82 according to the present embodiment. When the acquired data and the evaluation target information compared in the comparison unit 81 do not match, the updating unit 82 generates a new meta-ID that links with the acquired data. The updating unit 82 updates the meta ID estimation processing database by machine learning using the acquired data and the new meta ID generated, as new training data. The updating unit 82 stores the acquired data in the meta ID estimation processing database, as evaluation target information.

In addition, the updating unit 82 stores the new meta ID as a new content ID in the reference database, and stores the new content ID in the reference database in association with one item of the reference information stored in the reference database.

<Approval Unit 83>

The approval unit 83 assigns the first approval information to the combination of the newly stored evaluation target information and the meta ID, and stores this in the meta ID estimation processing database updated by the updating unit 82. In this case, the first approval time information, the first approver information, and the first approval meta information are stored together. Furthermore, the approval unit 83 may assign the first approval information to the combination of the newly memorized evaluation target information, the meta ID, and the degree of meta association, and stores this.

The approval unit 83 assigns second approval information to the combination of the new content ID and the reference information stored in the reference database, and stores this. In this case, the second approval time information, the second approver information, and the second approval meta information are stored together.

The present embodiment provides a comparison unit 81 that compares the acquired data with the evaluation target information, and an updating unit 82 that updates the first database by machine learning using the acquired data when the acquired data and the evaluation target information compared in the comparison unit 81 do not match, and the updating unit 82 generates a new meta ID that links with the acquired data, and, using the acquired data and the new meta ID generated, as new training data, updates the meta ID estimation processing database by machine learning. As a result of this, when acquired data is machine-learned as evaluation target information, machine learning can be executed using the newly generated meta ID that is small in capacity. Consequently, the task of updating the meta ID estimation processing database can be performed more easily.

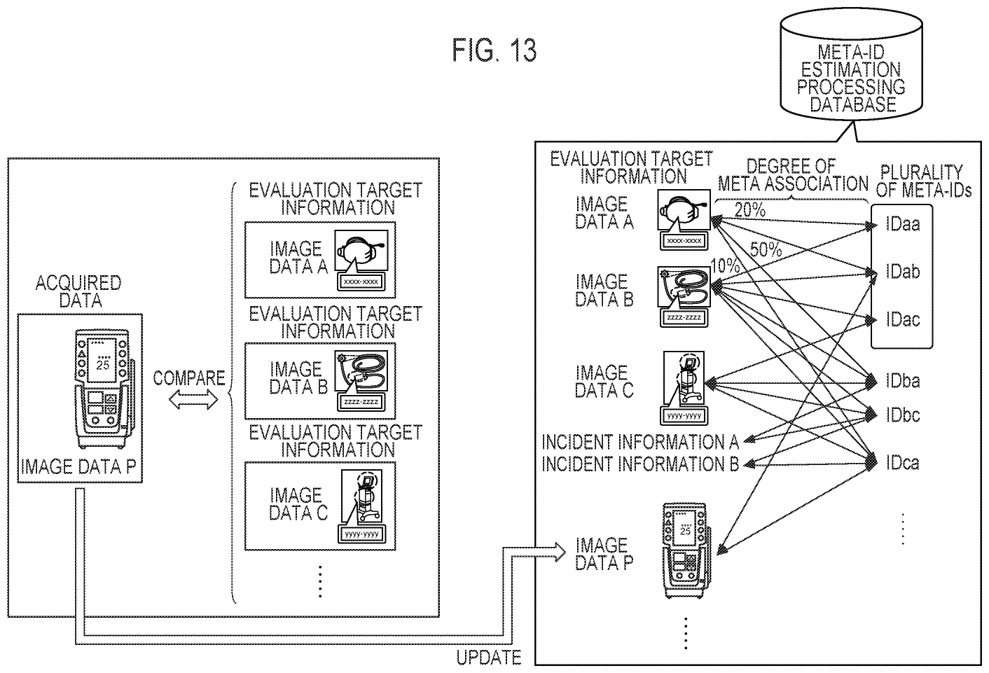

FIG. 13 is a schematic diagram to show a second example of the meta ID estimation processing database updated by the updating unit according to the present embodiment. When the acquired data and the evaluation target information compared in the comparison unit 81 do not match, the updating unit 82 may update the meta ID estimation processing database by machine learning using the acquired data and one of a plurality of meta IDs stored in the meta ID estimation processing database as new training data. At this time, the updating unit 82 may update the meta ID estimation processing database by machine learning using the acquired data and the first meta ID selected by the meta ID selection unit 12 as new training data.

The present embodiment provides a comparison unit 81 that compares the acquired data with the evaluation target information, and an updating unit 82 that updates the meta ID estimation processing database by machine learning using the acquired data, when the acquired data and the evaluation target information compared in the comparison unit 81 do not match, and the updating unit 82 updates the first database by machine learning using the acquired data and one of a plurality of meta IDs as new training data. As a result of this, the acquired data can be linked with existing meta IDs stored in the meta ID estimation processing database as evaluation target information. Consequently, the task of updating the first database can be performed more easily.

In particular, according to the present embodiment, the updating unit 82 updates the meta ID estimation processing database by machine learning using the acquired data and the first meta ID selected by the meta ID selection unit 12 as new training data. As a result of this, the acquired data can be linked with existing meta IDs stored in the meta ID estimation processing database as evaluation target information. Consequently, the task of updating the first database can be performed more easily. In particular, evaluation target information is associated with the first meta ID as acquired data, so that the accuracy of the selection of first meta IDs can be improved further with reference to the meta ID estimation processing database.

Although medical devices 4 have been illustrated with the above-described embodiment, the embodiment may be applied to nursing care devices apart from medical devices 4.

<Nursing Care Device>

In the event nursing care devices are used, the information providing system 100 is used by users such as nursing care practitioners, including caregivers who use nursing care devices. The information providing system 100 is intended for use for nursing care devices that are used primarily by nursing care practitioners such as caregivers. The information providing system 100 selects, from acquired data carrying image data of a nursing care device, first reference information that is suitable when a user to perform a task related to the nursing care device works on the task. The information providing system 100 can provide, for example, a manual of the nursing care device to the user, and, in addition, provide incident information related to the nursing care device to the user, for example. By this means, the user can learn the manual of the nursing care device, learn about the incidents related to the nursing care device, and so forth.

The nursing care devices, as used herein, include ones that relate to movement in indoor and outdoor environments, such as, for example, wheelchairs, walking sticks, slopes, handrails, walkers, walking aids, devices for detecting wandering elderly people with dementia, moving lifts, and so forth. The nursing care devices also include ones that relate to bathing, such as, for example, bathroom lifts, bath basins, handrails for bathtub, handrails in bathtub, bathroom scales, bathtub chairs, bathtub scales, bathing assistance belts, simple bathtubs, and so forth. The nursing care devices also include ones that relate to bowel movement, such as, for example, disposable diapers, automatic waste cleaning apparatus, stool toilet seat, and so forth. The nursing care devices also include ones that relate to bedding, such as, for example, nursing care beds including electric beds, bed pads, bedsore prevention mats, posture changers and so forth. The nursing care devices 4 not only include nursing care devices that are provided for in laws and regulations, but also include mechanical devices (beds, for example) and the like that are similar to nursing care devices in appearance and structures but are not provided for in laws and regulations. The nursing care devices 4 include welfare tools. The nursing care devices 4 may be ones for use at nursing care sites such as nursing care facilities, and include a nursing care-related information management systems that store information about care recipients and information about the staff in nursing care facilities.

Although embodiments of the present invention have been described above, these embodiments have been presented simply by way of example, and are not intended to limit the scope of the invention. These novel embodiments can be implemented in a variety of other forms, and various omissions, replacements, and changes can be made without departing from the spirit of the invention. These embodiments and examples of variations thereof are included in the scope and gist of the invention, and are also included in the invention described in claims and equivalents thereof.

### REFERENCE SIGNS LIST

1: information providing device
4: medical device
5: user terminal
6: server
7: public communication network
10: housing
11: acquiring unit
12: meta ID selection unit
13: content ID selection unit
14: reference information selection unit
15: input unit
16: output unit
17: memory unit
18: control unit
81: comparison unit
82: updating unit
83: approval unit
100: information providing system
101: CPU
102: ROM
103: RAM
104: storage unit
105: I/F
106: I/F
107: I/F
108: input part
109: output part
110: internal bus
S11: acquiring step
S12: meta ID selection step
S13: content ID selection step
S14: reference information selection step
S15: output step

1. An information providing system for selecting reference information that is suitable when a user to perform a task related to a medical device works on the task, the information providing system comprising:
acquiring means for acquiring acquired data comprising first image data, in which a specific medical device and a specific identification label for identifying the specific medical device are photographed;
a first database that is built on machine learning, using a data structure comprising a plurality of items of training data including evaluation target information including image data, and meta IDs that link with the evaluation target information;
meta ID selection means for looking up the first database and selecting a first meta ID, among the plurality of meta IDs, based on the acquired data;
a second database that stores content IDs that link with the meta IDs, and the reference information corresponding to the content IDs;
content ID selection means for looking up the second database and selecting a first content ID, among a plurality of content IDs, based on the first meta ID;
reference information selection means for looking up the second database and selecting first reference information, among a plurality of items of reference information, based on the first content ID; and
output means for outputting output information including the first reference information,
wherein the image data includes an image that shows the medical device and an identification label for identifying the medical device, and
wherein the output means outputs the output information including the first meta ID, the evaluation target information that has been used to select the first meta ID, and the first content ID that has been used to select the first reference information.

2. An information providing system for selecting reference information that is suitable when a user to perform a task related to a nursing care device works on the task, the information providing system comprising:
acquiring means for acquiring acquired data including first image data, in which a specific nursing care device and a specific identification label for identifying the specific nursing care device are photographed;
a first database that is built on machine learning, using a data structure comprising a plurality of items of training data including evaluation target information including image data, and meta IDs that link with the evaluation target information;
meta ID selection means for looking up the first database and selecting a first meta ID, among the plurality of meta IDs, based on the acquired data;
a second database that stores content IDs that link with the meta IDs, and the reference information corresponding to the content IDs;
content ID selection means for looking up the second database and selecting a first content ID, among a plurality of content IDs, based on the first meta ID;
reference information selection means for looking up the second database and selecting first reference information, among a plurality of items of reference information, based on the first content ID; and
output means for outputting output information including the first reference information,
wherein the image data includes an image that shows the nursing care device and an identification label for identifying the nursing care device, and
wherein the output means outputs the output information including the first meta ID, the evaluation target information that has been used to select the first meta ID, and the first content ID that has been used to select the first reference information.

3. The information providing system according to claim 1, wherein the first database stores first approval information that shows that the evaluation target information and the meta ID are approved,
wherein the second database stores second approval information that shows that the content ID and the reference information are approved, and
wherein the output means outputs the output information including the first approval information related to the first meta ID and the evaluation target information that has been used to select the first meta ID, and the second approval information related to the first reference information and the first content ID that has been used to select the first reference information.

4. The information providing system according to claim 3, wherein the first approval information includes at least one of:
first approval time information that shows a time the evaluation target information and the meta ID were approved,
first approver information that shows a person who approved the evaluation target information and the meta ID, and
first approval meta information that shows a reason the evaluation target information and the meta ID were approved, and
wherein the second approval information includes at least one of:
second approval time information that shows a time the content ID and the reference information were approved,
second approver information that shows a person who approved the content ID and the reference information, and
second approval meta information that shows a reason the content ID and the reference information were approved.

5. The information providing system according to claim 1, further comprising:
comparison means for comparing the acquired data with the evaluation target information; and
updating means for updating the first database by machine learning using the acquired data when the acquired data and the evaluation target information compared in the comparison means do not match,
wherein the updating means generates a new meta ID that links with the acquired data, and updates the first database by machine learning using the acquired data and the new meta ID generated, as new training data.

6. The information providing system according to claim 1, further comprising:
comparison means for comparing the acquired data with the evaluation target information; and
updating means for updating the first database by machine learning using the acquired data when the acquired data and the evaluation target information compared in the comparison means do not match,
wherein the updating means updates the first database by machine learning using the acquired data and any one of the plurality of meta IDs, as new training data.

7. The information providing system according to claim 6, wherein the updating means updates the first database by machine learning using the acquired data and the first meta ID selected by the meta ID selection means, as new training data.

8. The information providing system according to claim 2, wherein the first database stores first approval information that shows that the evaluation target information and the meta ID are approved,
wherein the second database stores second approval information that shows that the content ID and the reference information are approved, and
wherein the output means outputs the output information including the first approval information related to the first meta ID and the evaluation target information that has been used to select the first meta ID, and the second approval information related to the first reference information and the first content ID that has been used to select the first reference information.

9. The information providing system according to claim 8, wherein the first approval information includes at least one of:
first approval time information that shows a time the evaluation target information and the meta ID were approved,
first approver information that shows a person who approved the evaluation target information and the meta ID, and
first approval meta information that shows a reason the evaluation target information and the meta ID were approved, and
wherein the second approval information includes at least one of:
second approval time information that shows a time the content ID and the reference information were approved,
second approver information that shows a person who approved the content ID and the reference information, and
second approval meta information that shows a reason the content ID and the reference information were approved.

10. The information providing system according to claim 2, further comprising:
comparison means for comparing the acquired data with the evaluation target information; and
updating means for updating the first database by machine learning using the acquired data when the acquired data and the evaluation target information compared in the comparison means do not match,
wherein the updating means generates a new meta ID that links with the acquired data, and updates the first database by machine learning using the acquired data and the new meta ID generated, as new training data.

11. The information providing system according to claim 1, further comprising:
comparison means for comparing the acquired data with the evaluation target information; and
updating means for updating the first database by machine learning using the acquired data when the acquired data and the evaluation target information compared in the comparison means do not match,
wherein the updating means updates the first database by machine learning using the acquired data and any one of the plurality of meta IDs, as new training data.

12. The information providing system according to claim 11, wherein the updating means updates the first database by machine learning using the acquired data and the first meta ID selected by the meta ID selection means, as new training data.
